SEQUENCE LISTING (35 sequences)

SEQ ID NO: 1 - DNA, 405 bp, Aspergillus nidulans
  CDS (1)..(405)
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Aspergillus nidulans
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Aspergillus nidulans
  CDS (1)..(471)
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Aspergillus nidulans
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, unknown
  CDS (1)..(336): hydrophobin sequence with characteristic cysteine-pattern
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, unknown
  Hydrophobin sequence with characteristic cysteine-pattern
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, unknown
  CDS (1)..(357): hydrophobin sequence with characteristic cysteine-pattern
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, unknown
  hydrophobin sequence with characteristic cysteine-pattern
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Schizophyllum communae
  CDS (1)..(408)
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, Schizophyllum communae
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Artificial sequence
  CDS (1)..(483): Artificial hydrophobin sequence with characteristic cysteine-pattern
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Artificial sequence
  Artificial hydrophobin sequence with characteristic cysteine-pattern
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Artificial sequence
  CDS (1)..(465): Artificial hydrophobin sequence with characteristic cysteine-pattern
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Artificial sequence
  Artificial hydrophobin sequence with characteristic cysteine-pattern
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Bacillus subtilis
  CDS (1)..(882)
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, Bacillus subtilis
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Bacillus subtilis
  CDS (1)..(591)
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Bacillus subtilis
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Artificial sequence
  CDS (1)..(1329): fusion protein
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Artificial sequence
  fusion protein
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Artificial seqence
  CDS (1)..(1395): fusion protein
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Artificial sequence
  fusion protein
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Artificial sequence
  CDS (1)..(1407): fusion protein
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Artificial sequence
  fusion protein
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 28 bp, Artificial sequence
  Oligonucleotide Hal570
gcgcgcccatggctcaaacaggtactga

SEQ ID NO: 26 - DNA, 28 bp, Artificial sequence
  Oligonucleotide Hal571
gcagatctccagccgcgttcttgcatac

SEQ ID NO: 27 - DNA, 30 bp, Artificial sequence
  Oligonucleotide Hal572
ggccatgggattaacaataggtgtactagg

SEQ ID NO: 28 - DNA, 33 bp, Artificial sequence
  Oligonucleotide Hal573
gcagatcttacaagtgccttttgcttatattcc

SEQ ID NO: 29 - DNA, 38 bp, Artificial sequence
  Oligonucleotide KaM416
gcagcccatcagggatccctcagccttggtaccagcgc

SEQ ID NO: 30 - DNA, 49 bp, Artificial sequence
  Oligonucleotide KaM417
ccgtagctagtggatccattgaaggccgcatgaagttctccgtctccgc

SEQ ID NO: 31 - DNA, 45 bp, Artificial sequence
  Oligonucleotide KaM434
gctaagcggatccattgaaggccgcatgaagttctccattgctgc

SEQ ID NO: 32 - DNA, 30 bp, Artificial sequence
  Oligonucleotide KaM435
ccaatggggatccgaggatggagccaaggg

SEQ ID NO: 33 - DNA, 38 bp, Artificial sequence
  Oligonucleotide KaM418
ctgccattcaggggatcccatatggaggagggagacag

SEQ ID NO: 34 - DNA, 32 bp, Artificial sequence
  Oligonucleotide KaM464
cgttaaggatccgaggatgttgatgggggtgc

SEQ ID NO: 35 - DNA, 35 bp, Artificial sequence
  Oligonucleotide KaM465
gctaacagatctatgttcgcccgtctccccgtcgt

(19) United States
(12) Patent Application Publication
(10) Pub. No.: US 2008/0319168 A1
(43) Pub. Date: Dec. 25, 2008

(54) Method for Coating Surfaces with Hydrophobins

(75) Inventors: Thomas Subkowski, Ladenburg (DE); Marvin Karos, Schwetzingen (DE); Claus Bollschweiler, Heidelberg (DE); Ulf Baus, Dossenheim (DE); Patrick Rüdiger, Hassloch (DE); Michael Lang, Edingen-Neckarhausen (DE); Thorsten Montag, Osthofen (DE); Alexandra Kasprzyk, Bellheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/883,755

(22) PCT Filed: Feb. 7, 2006
(86) PCT No.: PCT/EP06/50723

§ 371 Date: Aug. 6, 2007

(30) Foreign Application Priority Data
Feb. 7, 2005 (DE) 10 2005 005 737.3
Feb. 17, 2005 (DE) 10 2005 007 480.4
Oct. 26, 2005 (DE) 10 2005 051 515.0

(51) Int. Cl. C07K 14/37; B05D 3/02

(52) U.S. Cl. 530/350; 427/372.2

(57) ABSTRACT

A method for coating surfaces with hydrophobin fusions at a pH of ≧4, and a surface having a coating which comprises at least one hydrophobin fusion.

The present invention relates to a method for coating surfaces with hydrophobin fusions at a pH of ≧4 and to surfaces having a coating which comprise hydrophobin fusions.

Hydrophobins are small proteins of from about 100 to 150 amino acids which are characteristic of filamentous fungi, for example Schizophyllum commune. As a rule, they possess 8 cystein units. Hydrophobins can be isolated from natural sources, for example.

Hydrophobins exhibit a pronounced affinity for interfaces and are therefore suitable for coating surfaces. Thus, Teflon, for example, can be coated with hydrophobins, resulting in a hydrophilic surface being obtained.

The prior art has proposed using hydrophobins for a variety of applications.

WO 96/41882 proposes using hydrophobins which are isolated from edible fungi as emulsifiers, thickeners or surface-active substances, for hydrophilizing hydrophobic surfaces, for improving the water resistance of hydrophilic substrates, and for preparing oil-in-water emulsions or water-in-oil emulsions. The document also proposes pharmaceutical applications such as the production of ointments or creams as well as cosmetic applications such as skin protection or the production of hair shampoos or hair rinses.

EP-B1 252 516 discloses the coating of windows, contact lenses, biosensors, medical devices, receptacles for carrying out experiments or for storage, ship holds, solid particles or frames or the bodywork of private cars with a solution comprising hydrophobins at a temperature of from 30 to 80° C. Preference is given to additionally using a surface-active substance as a coating aid. A type SC3 hydrophobin isolated from fungi (Schizophyllum commune) is used in the examples. Freeze-dried SC3 is used for pre-paring the coating solution, with the SC3 being dissolved in trifluoroacetic acid, the mixture being dried in a stream of nitrogen and the residue then being dissolved in water or a buffer solution. This procedure is laborious.

WO 2005/068087 proposes, as an alternative to the heating, coating in an acid pH range. The document discloses a method for coating surfaces with hydrophobins at a pH of less than 7, preferably less than 4 and particularly preferably less than 2. It additionally proposes a method for optimizing the coating conditions while varying the parameters pH, incubation time, concentration and the presence of a buffer. A type SC3 hydrophobin from natural sources is used in the examples.

The present application relates to coating surfaces with a novel class of hydrophobins which do not occur naturally. These hydrophobins are hydrophobin fusions in which naturally occurring hydrophobins are linked to peptide sequences which are at least 20 amino acids in length and which are not naturally linked to a hydrophobin. These hydrophobin fusions are also suitable for coating surfaces.

It has been found, surprisingly, that the quality of the coatings which are obtained using hydrophobin fusions does not decline even at elevated pH values. This behavior, which is the reverse of that of naturally occurring hydrophobins, makes it possible to obtain qualitatively high-grade coatings with hydrophobins in the alkaline range as well.

The following is to be stated with regard to the details of the invention:

“Hydrophobin fusions” which exhibit the following general structural formula (I)

Xₙ—C¹—X₁₋₅₀—C²—X₀₋₅—C³—X₁₋₁₀₀—C⁵—X₁₋₅₀—C⁶—X₀₋₅—C⁷—X₁₋₅₀—C⁸—Xₘ (I)

where X can be any of the 20 naturally occurring amino acids (Phe, Leu, Ser, Tyr, Cys, Trp, Pro, His, Gln, Arg, Ile Met, Thr, Asn, Lys, Val, Ala, Asp, Glu and Gly), are used for carrying out the present invention. X can in each case be identical or different in this connection. In the formula, the indices at each X constitute the number of amino acids and C is cysteine, alanine, serine, glycine, methionine or threonine, with at least four of the residues designated by C being cysteine. The indices n and m are, independently of each other, natural numbers of from 0 to 500, preferably of from 15 to 300, with the proviso that at least one of the peptide sequences designated by Xₙ and Xₘ is a peptide sequence which is at least 5, preferably at least 20 amino acids in length and which is not naturally linked to a hydrophobin.

The polypeptides formula according to (I) are furthermore characterized by the property that, at room temperature and after having coated a glass surface, they increase the contact angle of a water drop by at least 20°, preferably at least 25° and particularly preferably 30°, in each case compared with the contact angle of a water drop of the same size with the uncoated glass surface.

The amino acids designated by C¹ to C⁸ are preferably cysteines; however, they can also be replaced with other amino acids of similar space-filling, preferably with alanine, serine, threonine, methionine or glycine. However, at least four, preferably at least 5, particularly preferably at least 6 and in particular at least 7, of the C¹ to C⁸ positions should consist of cysteines. In the proteins which are used in accordance with the invention, cysteines can either be present in the reduced state or form disulfide bridges with each other. Particular preference is given to the intramolecular formation of C—C bridges particularly that involving the formation of at least one, preferably 2, particularly preferably 3, and very particularly preferably 4, intramolecular disulfide bridges. In the case of the above-described replacement of cysteines with amino acids of similar space-filling, those C positions which can form intramolecular disulfide bridges with each other are advantageously replaced in pairs.

If cysteines, serines, alanines, glycines, methionines or threonines are also used in the positions designated by X, the numbering of the individual C positions in the general formulae can change correspondingly.

Preference is given to hydrophobin fusions of the general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

for carrying out the present invention, where X, C and the indices at X and C have the above meaning, but the indices n and m stand for numbers between 0 and 300, and the proteins are still characterized by the abovementioned contact angle change, with the proviso that at least one of the peptide sequences designated by Xₙ and Xₘ is a peptide sequence which is at least 15, preferably at least 35, amino acids in length and which is not naturally linked to a hydrophobin.

Particular preference is given to using hydrophobin fusions of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

where X, C and the indices at X and C have the above meaning, the indices n and m stand for numbers between 0 and 200 and the proteins are still characterized by the abovementioned contact angle change, with the proviso that at least one of the peptide sequences designated by Xₙ and Xₘ is a peptide sequence which is at least 20 amino acids, preferably at least 50 amino acids, in length and which is not naturally linked to a hydrophobin, and at least 6 of the residues designated by C are still cysteine. Particular preference is given to all the C radicals being cysteine.

The residues which are not naturally linked to a hydrophobin will also be termed fusion partners in that which follows. This is intended to express the fact that the proteins can consist of at least one hydrophobin moiety and one fusion partner which do not occur together in this form in nature.

The fusion partner can be selected from a large number of proteins. It is also possible for several fusion partners to be linked to one hydrophobin moiety, for example at the amino terminus (Xₙ) and at the carboxy terminus (Xₘ) of the hydrophobin moiety. However, it is also possible for two fusion partner moieties, for example, to be linked to one position (Xₙ or Xₘ) on the hydrophobin.

Proteins which naturally occur in microorganisms, in particular in E. coli or Bacillus subtilis, are particularly suitable fusion partner moieties. Examples of such fusion partner moieties are the sequences yaad (SEQ ID NOs:15 and 16),

yaae (SEQ ID NOs:17 and 18) and thioredoxin. Fragments or derivatives of these said sequences which only comprise a part, for example from 70 to 99% preferably from 5 to 50% and particularly preferably from 10 to 40%, of the said sequences, or in which individual amino acids or nucleotides are altered as compared with the said sequence, with the percentage values in each case relating to the number of amino acids, are also very suitable.

In another preferred embodiment, the hydrophobin fusion also exhibits what is termed an affinity domain (affinity tag/affinity tail) in addition to the fusion partner as a group Xₙ or Xₘ. These affinity domains are, in a manner which is known in principle, anchor groups which are able to interact with certain complementary groups and are able to facilitate the working-up and purification of the proteins. Examples of these affinity domains comprise (His)ₖ, (Arg)ₖ, (Asp)ₖ, (Phe)ₖ or (Cys)ₖ groups, with k in general being a natural number of from 1 to 10. The affinity domain can preferably be a (His)ₖ group where k is from 4 to 6.

The hydrophobin fusions which are used in accordance with the invention can also be modified in their polypeptide sequence as well, for example by means of glycosylation or acetylation or else by means of chemical crosslinking, for example using glutardialdehyde.

An essential property of the fusion proteins which are used in accordance with the invention is the change in surface properties when the surfaces are coated with the fusion proteins. The change in the surface properties can be determined experimentally by measuring the contact angle of a water drop before and after coating the surface with the protein and determining the difference in the two measurements.

The skilled person knows in principle how to carry out contact angle measurements. The measurements relate to room temperature and to 5 μl water drops and to the use of small glass plates as substrate. The precise experimental conditions for an example of a suitable method for measuring the contact angle are described in the experimental section. Under the conditions given in the experimental section, the fusion proteins which are used in accordance with the invention possess the property of increasing the contact angle by at least 20°, preferably at least 25°, particularly preferably at least 30°, in each case compared with the contact angle of a water drop of the same size with the uncoated glass surface.

Hydrophobin fusions which are preferred for carrying out the present invention are those having a hydrophobin moiety of the dewA, rodA, hypA, hypB, sc3, basf1 or basf2 type, which types are characterized structurally in the sequence listing which follows. The hydrophobin moieties can also be only parts or derivatives of these types. Several, preferably 2 or 3, hydrophobin moieties of the same or different structure can also be linked to each other.

The fusion proteins having the polypeptide sequences depicted in SEQ ID NOs: 20, 22, 24, and also the encoding nucleic acid sequences, in particular the sequences as depicted in SEQ ID NOs: 19, 21, 23 are particularly suitable for carrying out the present invention. Proteins which are formed from the polypeptide sequences depicted in SEQ ID NO: 20, 22 or 24 by the substitution, insertion or deletion of at least one and up to 10, preferably 5, particularly preferably 5%, of all the amino acids and which still possess at least 50% of the biological property of the starting proteins are also particularly preferred embodiments. In this connection, the biological property of the proteins is understood as being the increase in the contact angle by at least 200 as has already been described.

The hydrophobin fusions which are used in accordance with the invention can be pre-pared chemically by known methods of peptide synthesis, for example by means of Merrifield's solid-phase synthesis.

The hydrophobin fusions are preferably prepared by means of recombinant methods in which a nucleic acid sequence, in particular DNA sequence, encoding the fusion partner and such a sequence encoding the hydrophobin moiety are combined such that the desired hydrophobin fusion is produced in a host organism by genetic expression of the combined nucleic acid sequence.

In this connection, host organisms (production organisms) which are suitable for said preparation method can be prokaryotes (including the Archaea) or eukaryotes, particularly bacteria including halobacteria and methanococci, fungi, insect cells, plant cells and mammalian cells, particularly preferably Escherichia coli, Bacillus subtilis, Bacillus megaterium, Aspergillus oryzea, Aspergillus nidulans, Aspergillus niger, Pichia pastoris, Pseudomonas spec., lactobacilli, Hansenula polymorpha, Trichoderma reesei, SF9 (or related cells) and others.

The invention also relates to the use of expression constructs which comprise, under the genetic control of regulatory nucleic acid sequences, a nucleic acid sequence which encodes a polypeptide which is used in accordance with the invention and also to vectors which comprise at least one of these expression constructs.

Constructs which are employed preferably comprise a promoter 5′-upstream of the given coding sequence and a terminator sequence 3′-downstream as well as, if appropriate, other customary regulatory elements, in each case operatively linked to the coding sequence.

“Operative linkage” is understood as meaning the sequential arrangement of promoter, coding sequence, terminator and, if appropriate, other regulatory elements such that each of the regulatory elements is able to fulfil its function in accordance with its intended use in connection with expressing the coding sequence.

Examples of sequences which can be operatively linked are targeting sequences and also enhancers, polyadenylation signals and the like. Other regulatory elements comprise selectable markers, amplification signals, origins of replication and the like. Examples of suitable regulatory sequences are described in Goeddel, Gene Expression Technology: Methods in Enzymology 185, Academic Press, San Diego, Calif. (1990).

In addition to these regulatory sequences, the natural regulation of these sequences can still be present upstream of the actual structural genes and, if appropriate, have been genetically altered such that the natural regulation has been switched off and the expression of the genes has been increased.

A preferred nucleic acid construct advantageously also comprises one or more of the enhancer sequences which have already been mentioned, which sequences are functionally linked to the promoter and enable the expression of the nucleic acid sequence to be increased. Additional advantageous sequences, such as further regulatory elements or terminators, can also be inserted at the 3′ end of the DNA sequences.

The nucleic acids can be present in the construct in one or more copies. The construct can comprise yet other markers, such as antibiotic resistances or genes which complement auxotrophies, for selecting for the construct, if appropriate.

Regulatory sequences which are advantageous for the method are present, for example, in promoters such as the cos, tac, trp, tet, trp-tet, lpp, lac, lpp-lac, laclq-T7, T5, T3, gal, trc, ara, rhaP(rhaPBAD) SP6, lambda-PR or imlambda-P promoter, which promoters are advantageously used in Gram-negative bacteria. Other advantageous regulatory sequences are present, for example, in the Gram-positive promoters amy and SP02, or in the yeast or fungal promoters ADC1, MFalpha, AC, P-60, CYC1, GAPDH, TEF, rp28 and ADH.

Artificial promoters can also be used for the regulation.

For being expressed in a host organism, the nucleic acid construct is advantageously inserted into a vector, such as a plasmid or a phage, which enables the genes to be expressed optimally in the host. Apart from plasmids and phages, vectors are also to be understood as being any other vectors which are known to the skilled person, that is, for example, viruses, such as SV40, CMV, baculovirus and adenovirus, transposons, IS elements, phasmids, cosmids and linear or circular DNA and also the Agrobacterium system.

These vectors can either replicate autonomously in the host organism or be replicated chromosomally. These vectors constitute another embodiment of the invention. Examples of suitable plasmids are pLG338, pACYC184, pBR322, pUC18, pUC19, pKC30, pRep4, pHS1, pKK223-3, pDHE19.2, pHS2, pPLc236, pMBL24, pLG200, pUR290, pIN-III″3-B1, tgt11 or pBdCl, in E. coli, plJ101, plJ364, plJ702 or plJ361, in Streptomyces, pUB110, pC194 or pBD214 in Bacillus, pSA77 or pAJ667, in Corynebacterium, pALS1, plL2 or pBB116 in fungi, 2alpha, pAG-1, YEp6, YEp13 or pEMBLYe23, in yeasts, or pLGV23, pGHlac+, pBIN19, pAK2004 or pDH51, in plants. Said plasmids constitute a small selection of the possible plasmids. Other plasmids are known to the skilled person and can be found, for example, in the book Cloning Vectors (Eds. Pouwels P. H. et al. Elsevier, Amsterdam-New York-Oxford, 1985, ISBN 0 444 904018).

Advantageously, the nucleic acid construct additionally comprises, for the purpose of expressing the other genes which are present, 3′- and/or 5′-terminal regulatory sequences which are intended for increasing expression and which are selected for optimal expression in dependence on the host organism and gene or genes which are chosen.

These regulatory sequences are intended to enable the genes to be expressed selectively and to enable the proteins to be expressed. Depending on the host organism, this can mean, for example, that the gene is only expressed or overexpressed following induction or that it is immediately expressed and/or overexpressed.

In this connection, the regulatory sequences or factors can preferably influence positively, and thereby increase, the gene expression of the inserted genes. Thus, the regulatory elements can advantageously be augmented at the transcriptional level by using strong transcription signals such as promoters and/or enhancers. However, in addition to that, it is also possible to augment the translation by, for example, improving the stability of the mRNA.

In another embodiment of the vector, the vector comprising the nucleic acid construct or the nucleic acid can also advantageously be introduced into the microorganisms in the form of a linear DNA and integrated into the genome of the host organism by way of heterologous or homologous recombination. This linear DNA can consist of a linearized vector, such as a plasmid, or only of the nucleic acid construct or the nucleic acid.

In order to achieve optimal expression of heterologous genes in organisms, it is advantageous to alter the nucleic acid sequences in accordance with the specific codon usage which is employed in the organism. The codon usage can be readily ascertained with the aid of computer analyses of other known genes of the organism concerned.

An expression cassette is prepared by fusing a suitable promoter with a suitable coding nucleotide sequence and a terminator signal or polyadenylation signal. To do this, use is made of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which enables the genes to be expressed optimally in the host. Vectors are well known to the skilled person and can be found, for example, in “Cloning Vectors” (Pouwels P. H. et al., Eds., Elsevier, Amsterdam-New York-Oxford, 1985).

The vectors can be used to prepare recombinant microorganisms which are transformed, for example, with at least one vector and can be used for producing the proteins which are used in accordance with the invention. Advantageously, the above-described recombinant constructs are introduced into a suitable host system and expressed in this system. In this connection, customary cloning and transfection methods which are known to the skilled person, such as coprecipitation, protoplast fusion, electroporation, retroviral transfection and the like, are preferably used in order to express said nucleic acids in the given expression system. Suitable systems are described, for example, in Current Protocols in Molecular Biology, F. Ausubel et al., Eds., Wiley Interscience, New York 1997, or Sambrook et al. Molecular Cloning: A Laboratory Manual, 2^{nd} edtn., Cold Spring Harbor Laboratory, Cold Spring Harbor Laboratory Press, Cold Spring Harbor, N.Y., 1989.

It is also possible to prepare homologously recombined microorganisms. A vector which comprises at least one segment of a gene or a coding sequence which is to be used in accordance with the invention in which, if appropriate, at least one amino acid deletion, addition or substitution has been introduced in order to alter, e.g. functionally disrupt, the sequence (thereby forming a knockout vector) is prepared for this purpose. The sequence which is introduced can, for example, also be a homolog from a related microorganism or be derived from a mammalian, yeast or insect source. The vector which is used for the homologous recombination can alternatively be constituted such that while the endogenous gene mutates, or is in some other way altered, in connection with homologous recombination, it still encodes the functional protein (e.g. the upstream regulatory region can be altered such that this alters the expression of the endogenous protein). The altered segment of the gene which is used in accordance with the invention is in the homologous recombination vector. The construction of vectors which are suitable for homologous recombination is described, for example, in Thomas, K. R. and Capecchi, M. R. (1987) Cell 51:503.

Any prokaryotic or eukaryotic organisms are in principle suitable for being used as recombinant host organisms for the nucleic acid or the nucleic acid construct which is used in accordance with the invention. Microorganisms such as bacteria, fungi or yeasts are advantageously used as host organisms. Gram-positive or Gram-negative bacteria, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium or Rhodococcus, are advantageously used.

The organisms which are used in the method for preparing hydrophobin fusions are grown or cultured in dependence on the host organism and in a manner known to the skilled person. Microorganisms are as a rule grown, at temperatures of between 0 and 100° C., preferably between 10 and 60° C., and while being gassed with oxygen, in a liquid medium which comprises a carbon source, usually in the form of sugars, a nitrogen source, usually in the form of organic nitrogen sources such as yeast extract or salts such as ammonium sulfate, trace elements such as iron, manganese and magnesium salts, and also, if appropriate, vitamins. In this connection, the pH of the nutrient liquid can or cannot be maintained at a fixed value, that is regulated during the growth. The growth can take place batch-wise, semibatch-wise or continuously. Nutrients can be introduced initially at the beginning of the fermentation or be subsequently fed in semicontinuously or continuously. The enzymes can be isolated from the organisms using the method described in the examples or be used for the reaction as a crude extract.

Fusion proteins, or functional biologically active fragments thereof, which are used in accordance with the invention can be prepared by means of a recombinant method in which a microorganism which produces proteins is cultured, the expression of the proteins is induced, if appropriate, and the proteins are isolated from the culture. The proteins can also be produced in this way on an industrial scale if desired. The recombinant microorganism can be cultured and fermented using known methods. Bacteria can, for example, be propagated in TB medium or LB medium and at a temperature of from 20 to 40° C. and a pH of from 6 to 9. Suitable culturing conditions are described in detail in, for example, T. Maniatis, E. F. Fritsch and J. Sambrook, Molecular Cloning: A Laboratory Manual, Cold Spring Harbor Laboratory, Cold Spring Harbor, N.Y. (1989).

If the proteins which are used in accordance with the invention are not secreted into the culture medium, the cells are then disrupted and the product is isolated from the lysate using known methods for isolating proteins. The cells can, as desired, be disrupted by high-frequency ultrasound, by high pressure, as, for example, in a French pressure cell, by osmolysis, by the action of detergents, lytic enzymes or organic solvents, by using homogenizers or by a combination of several of the methods cited.

The fusion proteins which are used in accordance with the invention can be purified by means of known chromatographic methods such as molecular sieve chromatography (gel filtration), such as Q-Sepharose chromatography, ion exchange chromatography and hydrophobic chromatography, as well as by means of other customary methods such as ultrafiltration, crystallization, salting-out, dialysis and native gel electrophoresis. Suitable methods are described, for example in Cooper, F. G., Biochemische Arbeitsmethoden [Biochemical Working Methods], Verlag Water de Gruyter, Berlin, New York, or in Scopes, R., Protein Purification, Springer Verlag, New York, Heidelberg, Berlin.

It can be particularly advantageous, for the purpose of facilitating isolation and purification, to provide the hydrophobin fusions with special anchoring groups which are able to bind to corresponding complementary groups on solid supports, in particular suitable polymers. These solid supports can, for example, be used as the filling for chromatography columns, and the efficiency of the separation can as a rule be markedly increased in this way. Such separation methods are also known as affinity chromatography. In order to incorporate the anchoring groups, it is possible, when preparing the proteins, to make use of vector systems or oligonucleotides which extend the cDNA by particular nucleotide sequences and thereby encode altered proteins or fusion proteins. For easier purification, modified proteins comprise what are termed tags which function as anchors, for example the modification which is known as a hexahistidine anchor. Hydrophobin fusions which are modified with histidine anchors can, for example, be purified chromatographically using nickel-Sepharose as the column filling. The hydrophobin fusion can then be eluted from the column once again using suitable agents, such as an imidazole solution, for the elution.

Working-up methods can naturally also be combined with each other. For example, it is possible to initially separate by means of chromatography and then to use dialysis to purify the resulting solution of substances which were used for the elution.

In a simplified purification method, it is possible to dispense with the chromatographic purification. For this, the cells are initially separated off from the fermentation broth using a suitable method, for example by means of microfiltration or by means of centrifugation. The cells can then be disrupted using suitable methods, for example using the methods which have already been mentioned above, and the cell debris can be separated from the inclusion bodies. The latter step can advantageously be effected by means of centrifugation. Finally, the inclusion bodies can be disrupted in a manner which is known in principle in order to release the hydrophobin fusions. This can be effected, for example, using acids, bases and/or detergents. As a rule, the inclusion bodies containing the hydrophobin fusions which are used in accordance with the invention can be dissolved completely within approx. 1 h by using 0.1 M NaOH. As a rule, the purity of the hydrophobin fusions which have been obtained using this simplified method is from 60 to 80% by weight based on the quantity of all the proteins. The solutions which are obtained in accordance with the simplified purification method which has been described can be used without further purification for coating surfaces. As a rule, the minor components do not interfere and at most only have a marginal effect on the coating result.

The concentration of hydrophobin fusions in the resulting hydrophobin solutions is usually from 0.1 mg/ml to 50 mg/ml.

The hydrophobin fusions can also be isolated from the solutions as a solid. This can, for example, be effected by freeze-drying or spray-drying in a manner which is known in principle.

In a preferred embodiment of the invention, the isolation can be effected by means of spray drying. While the spray drying can be performed using the chromatographically purified solution, preference is also given to using the solutions which are obtained by processing the inclusion bodies in accordance with the simplified purification method.

The solutions can, if appropriate, be neutralized for the purpose of carrying out the spray drying. A pH range of from 7 to 9 has been found to be particularly advantageous.

It is furthermore advisable, as a rule, to concentrate the starting solutions to a certain degree. A solid concentration in the starting solution of up to 30% by weight has been found to be useful. In general, a solids proportion of >5% leads to a finely powdered product. After that, the solution can be spray dried in a manner which is known in principle. Suitable appliances for the spray drying are available commercially. The optimum spray drying conditions vary with the appliance type and the sought-after throughput. Entry temperatures of from 130 to 180° C. and exit temperatures of from 50 to 80° C. have been found to be advantageous in the case of hydrophobin solutions. Auxiliary sub-stances such as sugar, mannitol, dextran or maltodextrin can optionally be used for the spray drying. A quantity of from 0 to 30% by weight, preferably of from 5 to 20% by weight, of these auxiliary substances, based on hydrophobin, has been found to be of value.

A formulation (F) which comprises at least water or an aqueous solvent mixture, and a hydrophobin fusion, is used for carrying out the coating method according to the invention with.

Suitable aqueous solvent mixtures comprise water and one or more solvents which are miscible with water. The choice of these components is only restricted insofar as the hydrophobin fusions and the other components have to be sufficiently soluble in the mixture. As a rule, these mixtures comprise at least 50% by weight, preferably at least 65% by weight, and particularly preferably at least 80% by weight of water. Very particular preference is given to only using water. The skilled person will make a suitable selection from the water-miscible solvents depending on the desired properties of the formulation F. Examples of suitable water-miscible solvents comprise monoalcohols, such as methanol, ethanol or propanol, higher alcohols, such as ethylene glycol or polyether polyols, and also ether alcohols, such as butyl glycol or methoxypropanol.

According to the invention, the formulation which is used for the treatment has a pH of ≧4, preferably ≧6 and particularly preferably ≧7. For example, the pH can be 4, 5, 6, 7, 8, 9, 10 or 11. In particular, the pH is in the range of from 4 to 11, preferably of from 6 to 10, particularly preferably of from 7 to 9.5 and very particularly preferably of from 7.5 to 9. For example, the pH can be from 7.5 to 8.5 or from 8.5 to 9.

The formulation preferably comprises a suitable buffer for adjusting the pH. The skilled person will select a suitable buffer depending on the pH range which is envisaged for the coating. Buffers which may be mentioned are, for example, potassium dihydrogen phosphate buffer, tris(hydroxymethyl)aminomethane buffer (Tris buffer), borax buffer, sodium hydrogen carbonate buffer or sodium hydrogen phosphate buffer. Tris buffer is preferred.

The skilled person will determine the concentration of the buffer in the solution in dependence of the desired properties of the formulation. As a rule, the skilled person will take care to ensure that the buffering capacity is adequate in order to obtain coating conditions which are as constant as possible. A concentration of from 0.001 mol/l to 1 mol/l, preferably of from 0.005 mol/l to 0.1 mol/l, and particularly preferably of from 0.01 mol/1 to 0.05 mol/l, has proved to be of value.

The formulation additionally comprises at least one hydrophobin fusion. Hydrophobin fusions and preferred hydrophobin fusions were already specified at the outset. It is naturally also possible to use mixtures of different hydrophobin fusions. The hydrophobin fusion yaad-Xa-dewA-his (SEQ ID NO: 20), or proteins derived therefrom in which the fusion partner yaad is truncated, are particularly suitable for carrying out the pre-sent invention.

The concentration of the hydrophobin fusions in the solution will be chosen by the skilled person in dependence on the desired properties of the coating. As a rule, a more rapid coating can be achieved with higher concentrations. As a rule, a concentration of from 0.1 μg/ml to 1000 μg/ml, preferably of from 1 μg/ml to 500 μg/ml, particularly preferably of from 10 μg/ml to 250 μg/ml, very particularly preferably of from 30 μg/ml to 200 μg/ml, and, for example, of from 50 to 100 μg/ml, has proved to be of value.

In addition to this, the formulation F can optionally comprise further components or additives.

Examples of additional components comprise surfactants. Examples of suitable surfactants are nonionic surfactants which comprise polyalkoxy groups, in particular polyethyllene oxide groups. Examples comprise polyoxyethylene stearates, alkoxylated phenols and the like. Other examples of suitable surfactants comprise polyethylene glycol(20)sorbitan monolaurate (Tween® 20), polyethylene glycol(20)sorbitan monopalmitate (Tween® 40), polyethylene glycol(20)sorbitan monostearate (Tween® 60), polyethylene glycol(20)sorbitan monooleate (Tween® 80), cyclohexylmethyl-β-D-maltoside, cyclohexylethyl-β-D-maltoside, cyclohexyl-n-hexyl-β-D-maltoside, n-undecyl-β-D-maltoside, n-octyl-β-D-maltopyranoside, n-octyl-β-D-glucopyranoside, n-octyl-α-D-glucopyranoside and n-dodecanoylsucrose. Other surfactants are disclosed, for example, in WO 2005/68087 page 9, line 10, to page 10, line 2. The concentration of surfactants is as a rule from 0.001% by weight to 0.5% by weight, preferably from 0.01% by weight to 0.25% by weight, and particularly preferably from 0.1% by weight to 0.2% by weight, in each case based on the quantity of all the components in the formulation.

Furthermore, metal ions, in particular divalent metal ions, can also be added to the formulation. Metal ions can contribute to a more uniform coating. Examples of suitable divalent metal ions comprise, for example, alkaline earth metal ions such as Ca²⁺ ions. These metal ions can preferably be added as salts which are soluble in the formulation, for example in the form of chlorides, nitrates or carbonate, acetate, citrate, gluconate, hydroxide, lactate, sulfate, succinate or tartrate. For example, CaCl₂ or MgCl₂ can be added. The solubility can also optionally be increased by means of suitable auxiliaries, for example complexing agents. If present, the concentration of these metal ions is as a rule from 0.01 mmol/l to 10 mmol/l, preferably from 0.1 mmol/l to 5 mmol/l and particularly preferably from 0.5 mmol/l to 2 mmol/l.

The additional components can furthermore also include naturally occurring hydrophobins which are employed in the mixture together with the hydrophobin fusions.

It is possible in principle to use those solutions which accrue in connection with preparing or working up the hydrophobins for preparing the formulations F. In this connection, the hydrophobins can either be chromatographically purified hydrophobin fusions or else solutions which are obtained by terminating the inclusion bodies. These solutions can, in addition to the hydrophobin fusion, also comprise other components from the workup, for example buffers, residues of the auxiliaries used for the elution or auxiliary substances from the spray drying. These components do not need to be removed provided they do not interfere with the coating process.

As a rule, the solutions from the workup exhibit a markedly higher hydrophobin concentration than is required for the coating. They can be diluted to the desired concentration by adding water, other water-miscible solvents or buffer solutions.

In a preferred embodiment of the method, solid hydrophobin fusions, preferably the abovementioned hydrophobin fusions which are prepared by spray drying, are used for preparing the formulation F. Particularly advantageously, the spray-dried hydrophobin fusion can be readily dissolved in water or in the aqueous solvent mixture. This is a marked advantage as compared with solid, naturally occurring hydrophobins which, according to the prior art, have to be dissolved using trifluoroacetic acid (TFA) or formic acid. However, TFA/formic acid is undesirable for coating a number of substrates, which means that the TFA or formic acid has to be removed once again in an elaborate manner after the hydrophobin has been dissolved.

Other components can be dissolved in the formulation by, for example, simply stirring them in. It is naturally also possible to previously dissolve additional components and then to combine the solutions. Different spray-dried materials can be mixed before being dissolved. The spray-dried hydrophobin fusion can also, in a further step, be provided with additional components by, for example, spraying on other compounds and then drying. Conversely, a hydrophobin fusion can also be applied to already existing particles of auxiliary substances. It is likewise possible to modify the spray-dried hydrophobin, for example in the form of granulation.

In accordance with the invention, the surface to be coated is, for the coating, treated with the formulation.

In this connection, there is no restriction on the choice of the surfaces. These surfaces can be either smooth surfaces or surfaces having a pronounced surface structure. The surfaces can, for example, be the surfaces of molded articles, such as panels, films or the like. The surfaces can, for example, be composed of plastics such as Teflon, polyethylene, polypropylene, polystyrene, polymethyl methacrylate or other polymeric materials, of metals such as steel, aluminum, zinc, tin, copper or metal alloys such as brass, of natural or altered natural materials such as leather, textiles (e.g. cotton), paper and surfaces which are relevant for cosmetics (e.g. skin, hair, teeth or mucous membranes), of glass or of ceramic materials. Objects which are to be coated can also possess surfaces composed of different materials, for example combinations of glass, metal and plastics.

The surfaces to be coated can also, for example, be the surfaces of finely divided inorganic or organic substances, in particular inorganic or organic pigments or, for example, latex particles as well. Examples comprise typical paint or effect pigments or else typical fillers.

The skilled person will choose the method for treating the surface in dependence on the nature of the surface. For example, the object to be coated can be immersed in the formulation or the formulation can be applied to the surface by spraying on. This type of surface treatment is suitable for both planar and irregularly shaped surfaces. Sheet-like molded bodies such as panels or films can furthermore also be advantageously treated by coating or roller application. Excess formulation can be removed once again by means of suitable methods, for example by means of doctoring-off or squeezing. The coating can particularly preferably be performed by means of spraying. The skilled person is familiar with suitable spraying appliances.

Finely divided pigments and/or fillers can advantageously be coated by first of all dispersing the pigments in a suitable solvent and then, for the coating, adding the hydrophobin fusions, and optionally other auxiliary substances, to this dispersion. The pigment dispersions which are used can also advantageously be dispersions which accrue in connection with the wet-chemical preparation of pigments without the pigments having to be separated off beforehand provided other substances which are present in the dispersion do not interfere with the coating process.

As a rule, a certain exposure time is required for the hydrophobin fusions to settle on the surface. The skilled person will choose a suitable exposure time in dependence on the desired result. Examples of typical exposure times are from 0.1 to 12 h, without the invention having to be restricted to these times.

As a rule, the exposure time depends on the temperature and on the concentration of the hydrophobin fusion in the solution. The higher the temperature and the higher the concentration during the course of the coating process, the shorter the exposure time can be. The temperature during the course of the coating process can be room temperature or else it can be an elevated temperature. For example, possible temperatures are 5, 10, 20, 30, 40, 50, 60, 70, 80, 90, 100, 110 or 120° C. The temperature is preferably from 15 to 120° C., particularly preferably from 20 to 100° C., and, for example, from 40 to 100° C. or from 70 to 90° C. The temperature can be introduced, for example, by heating the bath in which the object to be coated is immersed. However, it is also possible to heat an immersed object subsequently, for example using IR radiation emitters. In the case of pigment dispersions, the dispersion can be heated.

After the coating, solvent which is still present in the coating is removed from the coating. This can be effected, for example, by means of simple evaporation in air. However, the removal of the solvent can also be facilitated by raising the temperature and/or using suitable gas flows and/or applying a vacuum. The evaporation can be facilitated by, for example, heating coated objects in a drying oven or blowing a heated gas flow onto them. The methods can also be combined, for example by drying in a circulating drying oven or a drying channel. Furthermore, the coating can, for the purpose of removing the solvent, also be heated by means of radiation, in particular IR radiation. Any type of broad-band IR radiation emitter, for example NIR, MIR or NIR radiation emitters can be used for this purpose. However, it is also possible, for example, to use IR lasers. These radiation sources are available commercially in a variety of radiation geometries. Pigment dispersions can, for example, also be dried by means of spray drying.

The skilled person will determine the temperature and the drying time during the course of the drying. A drying temperature of from 30 to 130° C., preferably of from 50 to 120° C., particularly preferably of from 70 to 110° C., very particularly preferably of from 75 to 105° C., and, for example, from 85 to 100° C., has proved to be of value. That which is meant here is the temperature of the coating itself. The temperature in a dryer can, of course, also be higher. The drying time is naturally inversely proportional to the drying temperature.

The temperature treatment during the course of the coating, and the drying, can advantageously be combined with each other. Thus, a surface can, for example, be initially treated with the formulation F at room temperature and subsequently dried and tempered at elevated temperatures. In a preferred embodiment of the method, an elevated temperature is applied at least in one of the two “treatment” and “drying” steps. A temperature which is higher than room temperature is preferably applied in both steps.

By using the method according to the invention to treat the surface, it is possible to obtain a surface which is coated with hydrophobin fusions and which comprises the material of the surface and also a layer which is located immediately on top of it and which exhibits at least one hydrophobin fusion and, if appropriate, other constituents of the formulation. In this connection, the entire surface, or only a part of the surface, can be covered with hydrophobin. The quality can be assessed by means of a variety of methods, for example by means of the contact angle measurement which has already been mentioned. The contact angle changes markedly as when coating with naturally occurring hydrophobins. Other methods are known to the skilled person from the prior art which was cited at the outset (e.g. “AFM” atomic force microscopy for directly detecting the protein layer on the surface).

The hydrophobin fusion layer can be subjected to further chemical modification before or after removing the solvent. It is, for example, possible to crosslink the layer using suitable crosslinkers. Examples of suitable crosslinkers comprise glutaraldehyde, formaldehyde and also other homobifunctional and heterobifunctional protein crosslinkers which are known from protein chemistry. This can therefore increase the stability of the layer. In addition, the binding to the substrate can be additionally improved in the case of protein-containing substrates such as leather and certain textiles and also in the case of surfaces which are relevant for cosmetics. The crosslinking can, for example, be performed by, after the coating, treating the layer containing the hydrophobin fusion with a second solution containing the crosslinker and then subsequently drying. It is furthermore also possible to pretreat protein-containing substrates, or other substrates, such that protein-reactive functional groups are formed on the surface of the substrate. While the abovementioned crosslinkers can, for example, be used for this purpose, it is also possible to use other chemicals such as ozone, peroxides or aldehydes. Another possibility consists in a coupling or augmentation of the coupling by way of metal ions. Appropriate protein sequences having affinity for metal ions are known to the skilled person (e.g. His₆ for Ni, Co, Fe, etc.) and can be attached to the hydrophobins using standard molecular biological techniques or coupling as used in protein chemistry. In this connection, the metal ions can be coupled beforehand to the surface to be coated or be used simultaneously with the hydrophobin coupling.

The following examples are intended to illustrate the invention in more detail:

Section A) Preparing the Hydrophobin Fusions which are Used in Accordance with the Invention

### EXAMPLE 1

### Preliminary Work for Cloning yaad-His₆/yaaE-His₆

A polymerase chain reaction was carried out using the oligonucleotides Hal570 and Hal571 (Hal 572/Hal 573). Genomic DNA from the bacterium Bacillus subtilis was used as template DNA. The resulting PCR fragment contained the coding sequence of the Bacillus subtilis yaaD/yaaE gene and an NcoI and a BglII restriction cleavage site at the respective ends. The PCR fragment was purified and cut with the restriction endonucleases NcoI and BglII. This DNA fragment was used as an insert and cloned into the Qiagen vector pQE60, which had been previously linearized with the restriction endonucleases NcoI and BglII. The vectors which were formed in this way, i.e. pQE60YMD#2/pQE60YaaE#5, can be used for expressing proteins consisting of YMD::HIS₆ and, respectively, YAAE::HIS₆.

HaI570: gcgcgcccatggctcaaacaggtactga
HaI571: gcagatctccagccgcgttcttgcatac
HaI572: ggccatgggattaacaataggtgtactagg
HaI573: gcagatcttacaagtgccttttgcttatattcc

### EXAMPLE 2

### Cloning yaad-Hydrophobin DewA-His₆

A polymerase chain reaction was carried out using the oligonucleotides KaM 416 and KaM 417. Genomic DNA from the mold Aspergillus nidulans was used as template DNA. The resulting PCR fragment contained the coding sequence of the hydrophobin gene dewA and an N-terminal faktor Xa proteinase cleavage site. The PCR fragment was purified and cut with the restriction endonuclease BamHI. This DNA fragment was used as an insert and cloned into the vector pQE60YMD#2, which had been previously linearized with the restriction endonuclease BglII.

The vector which was formed, i.e. #508 can be used for expressing a fusion protein consisting of YMD::Xa::dewA::HIS₆.

KaM416:
GCAGCCCATCAGGGATCCCTCAGCCTTGGTACCAGCGC
KaM417:
CCGTAGCTAGTGGATCCATTGAAGGCCGCATGAAGTTCTCCGTCTCCGC

### EXAMPLE 3

### Cloning yaad-Hydrophobin RodA-His₆

The plasmid #513 was cloned in analogy with plasmid #508 using the oligonucleotides KaM 434 and KaM 435.

KaM434:
GCTAAGCGGATCCATTGAAGGCCGCATGAAGTTCTCCATTGCTGC
KaM435:
CCAATGGGGATCCGAGGATGGAGCCAAGGG

### EXAMPLE 4

### Cloning yaad-Hydrophobin BASF1-His₆

The plasmid #507 was cloned in analogy with plasmid #508 using the oligonucleotides KaM 417 and KaM 418.

An artificially synthesized DNA sequence, i.e. hydrophobin BASF1, was used as template DNA (see annex).

KaM417:
CCCGTAGCTAGTGGATCCATTGAAGGCCGCAT-GAAGTTCTCCGTCTCCG
C
KaM418:
CTGCCATTCAGGGGATCCCATATGGAGGAGGGAGACAG

### EXAMPLE 5

### Cloning yaad-Hydrophobin BASF2-His₆

The plasmid #506 was cloned in analogy with plasmid #508 using the oligonucleotides KaM 417 and KaM 418.

An artificially synthesized DNA sequence, i.e. hydrophobin BASF2e, was used as template DNA (see annex).

KaM417:
CCCGTAGCTAGTGGATCCATTGAAGGCCGCAT-GAAGTTCTCCGTCTCCG
C
KaM418:
CTGCCATTCAGGGGATCCCATATGGAGGAGGGAGACAG

### EXAMPLE 6

### Cloning yaad-Hydrophobin SC3-His₆

The plasmid #526 was cloned in analogy with plasmid #508 using the oligonucleotides KaM464 and KaM465.

cDNA from Schyzophyllum commune was used as template DNA (see annex).

KaM464: CGTTAAGGATCCGAGGATGTTGATGGGGGTGC
KaM465: GCTAACAGATCTATGTTCGCCCGTCTCCCCGTCGT

### EXAMPLE 7

### Fermenting the Recombinant E. coli Strain Yaad-Hydrophobin DewA-His₆

3 ml of LB liquid medium are inoculated, in a 15 ml Greiner tube, with an E. coli strain which is expressing yaad-hydrophobin DewA-His₆. The medium is incubated at 37° C. for 8 h on a shaker rotating at 200 rpm. In each case 2×1 l baffled Erlenmeyer flasks containing 250 ml of LB medium (+100 μg of ampicillin/ml) are inoculated with in each case 1 ml of the preliminary culture and incubated at 37° C. for 9 h on a shaker which is rotating at 180 rpm.

13.5 l of LB medium (+100 μg of ampicillin/ml) are inoculated, in a 20 l fermenter, with 0.5 l of preliminary culture (OD₆₀₀ nm 1:10 measured against H₂O). 140 ml of 100 mM IPTG are added at an OD₆₀ₙₘ of ˜3.5. After 3 h, the fermenter is cooled down to 10° C. and the fermentation broth is centrifuged. The cell pellet is used for the further purification.

### EXAMPLE 8

### Purifying the Recombinant Hydrophobin Fusion Protein

(Purifying Hydrophobin Fusion Proteins which Possess a C-Terminal His6 Tag)

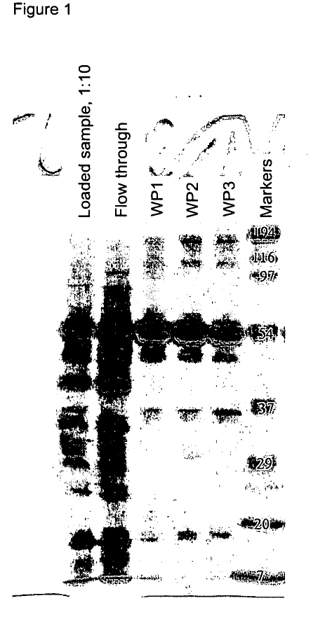

100 g of cell pellet (100-500 mg of hydrophobin) are made to a total volume of 200 ml with 50 mM sodium phosphate buffer, pH 7.5, and resuspended. The suspension is treated for 10 minutes with an Ultraturrax type T25 (Janke and Kunkel; IKA-Labortechnik) and then incubated at room temperature for 1 hour with 500 units of Benzonase (Merck, Darmstadt; order No. 1.01697.0001) for the purpose of degrading the nucleic acids. Prior to the cell disruption, filtration is carried out using a glass cartridge (P1). Two homogenizer runs at 1500 bar are carried out for the cell disruption and for shearing the remaining genomic DNA (M-110EH microfluidizer; Microfluidics Corp.). The homogenate is centrifuged (Sorvall RC-5B, GSA rotor, 250 ml centrifuge bottles, 60 minutes, 4° C., 12 000 rpm, 23 000 g), after which the supernatant is placed on ice and the pellet is resuspended in 100 ml of sodium phosphate buffer, pH 7.5. The centrifugation and resuspension are repeated three times, with the sodium phosphate buffer comprising 1% SDS during the third repeat. After the resuspension, the mixture is stirred for an hour and a final centrifugation is carried out (Sorvall RC-5B, GSA rotor, 250 ml centrifuge bottles, 60 minutes, 4° C., 12 000 rpm, 23 000 g). SDS-PAGE analysis indicates that the hydrophobin is present in the supernatant after the final centrifugation (FIG. 1). The experiments show that the hydrophobin is probably present in the form of inclusion bodies in the corresponding E. coli cells. 50 ml of the hydrophobin-comprising supernatant are loaded to a 50 ml nickel-Sepharose High Performance 17-5268-02 column (Amersham) which has been equilibrated with 50 mM Tris-Cl, pH 8.0, buffer. The column is washed with 50 mM Tris-Cl, pH 8.0, buffer and the hydrophobin is then eluted with 50 mM Tris-Cl, pH 8.0, buffer, which comprises 200 mM imidazole. The solution is dialyzed against 50 mM Tris-Cl, pH 8.0, buffer in order to remove the imidazole.

FIG. 1 shows the purification of the hydrophobin fusion which was prepared:

| | |
|---|---|
| Lane 1: | solution loaded on nickel-Sepharose column (diuted 1:10) |
| Lane 2: | flow through = washing step eluate |
| Lanes 3-5: | OD 280 maxima of the elution fractions |

The hydrophobin fusion in FIG. 1 has a molecular weight of approx. 53 kD. Some of the smaller bands represent breakdown products of the hydrophobin.

### EXAMPLE 9

### Simplified Purification Method

The E coli cell pellet obtained in Example 7 is pressed, in water, through a nozzle at 1000 bar. In connection with this, the cells are completely disrupted. Centrifugation is used to separate the hydrophobin, which has accrued in inclusion bodies, from the remaining cell debris. At a g value of 5000, 2 phases separate after 30 minutes. The lower, hydrophobin fusion-comprising phase is suspended once again with water and centrifuged as above. The inclusion bodies are then incubated for 60 minutes in 0.1 M NaOH and in this way dissolved completely. The pH is adjusted to 8 with phosphoric acid and the protein concentration is adjusted to 20 mg/ml. The purity (based on total protein) of the hydrophobin fusion which is produced in this way is 70%.

### EXAMPLE 10

### Spray Drying Hydrophobin

The hydrophobin solution which is obtained in Example 9 is subjected to further processing in a commercially available spray dryer.

The spray drying is effected in the added presence of 10% w/w mannitol and using an entry temperature of 160° C. and an exit temperature of 70° C. A finely powdered product was obtained.

### EXAMPLE 11

### Application Technology Test; Characterizing the Hydrophobin Fusion by the Change in the Contact Angle of a Water Drop on Glass

### Substrate:

Glass (window glass, Süddeutsche Glas [South German Glass, Mannheim):
Hydrophobin which has been spray dried as described in Example 10 is taken up in an aqueous buffer solution (50 mM Tris, pH 8+0.1 mM CaCl₂ (final concentration)+0.1% polyoxyethylene(20)sorbitan monolaureate (Tween® 20)) and adjusted to a concentration of 100 μg/mL
Small glass plates are incubated overnight (temperature 80° C.) and, after that, the coating is washed in distilled water
After that, an incubation is carried out, at 80° C. for 10 min, with 1% sodium dodecyl sulfate (SDS) solution in dist. water
Washing is carried out in dist. water

The samples are dried in air and the contact angle (in degrees) of a 5 μl drop of water is determined at room temperature.
The contact angle measurement was carried out on a Dataphysics Contact Angle System OCA 15+, software SCA 20.2.0. (November 2002) appliance. The measurement was carried out in accordance with the manufacturer's instructions.

Untreated glass gave a contact angle of 30±5°; the coated glass gave a contact angle of 75±5°.

### EXAMPLE 12

Using spraying for carrying out coating experiments with the hydrophobin fusion:

### 1. Spraying Polyethylene Plates:

A solution of yaad-Xa-dewA-his (SEQ ID NO: 20), which was obtained as described in Example 8, was used for the experiments. The solution also comprised sodium phosphate buffer at a concentration of 50 mM. The concentration of the hydrophobin fusion in the solution was 11.23 mg/ml while the pH of the solution was 7.5.

The solution which was obtained using the simplified purification method as described in Example 9 was also used.

For the spraying experiments, the solutions were diluted about 100-fold down to a concentration of 100 μg of hydrophobin fusion/ml. The following solutions or solvents were used for the diluting in each case:

| Example | | | pH of the |
|---|---|---|---|
| No. | Hydrophobin | Solution | solution |
| 10-1 | chromatographically | only water | 8.0-8.5 |
| | purified (Ex. 8) | | |
| 10-2 | chromatographically | Tris buffer (50 mM) | 7.5-8.0 |
| | purified (Ex. 8) | | |
| 10-3 | chromatographically | 0.1% by weight of | 7.5-8.0 |
| | purified (Ex. 8) | nonionic surfactant | |
| | | in water, (polyoxy- | |
| | | ethylene(20)sorbitan | |
| | | monolaurate, Tween ® 20) | |
| 10-4 | chromatographically | (0.1% by weight) | 4.0-4.5 |
| | purified (Ex. 8) | polyamide derivative in | |
| | | water (Lurotex ® A 25) | |
| 10-5 | chromatographically | (0.1% by weight) anionic | 8.5-9.0 |
| | purified (Ex. 8) | surfactant in water | |
| | | (Leophen ® M) | |
| 10-6 | from disrupted | only water | 8.0-8.5 |
| | material (Ex. 9) | | |
| 10-7 | from disrupted | Tris buffer (50 mM) | 7.5-8.0 |
| | material (Ex. 9) | | |

These solutions 10-1 to 10-5 were now sprayed, using a laboratory spraying appliance (Desaga SG1) onto polyethylene plates (Simona® PE-HWU) such that a thin, uniform film was formed on the surface. This liquid film dried completely within 2 h at RT. After a resting time of a further 2 hours, the plates were carefully rinsed with a large quantity of water and dried overnight in air.

In order to assess the quality, the contact angle of the coated surface was measured as described above. The ability of water to form a film on the surface was also assessed optically. The results are compiled in Table 1.

TABLE 1
Coating of polyethylene plates with hydrophobin fusions

| | | Contact | Formation of a |
|---|---|---|---|
| Solution | Hydrophobin | angle | film by water |
| Comparison: | — | 90° | no |
| untreated | | | |
| polyethylene | | | |
| Comparison: only | — | 90° | no |
| water | | | |
| Comparison: only | — | 90° | no |
| TRIS buffer | | | |
| 10-1 (water) | 100 μg/ml | 73° | yes |
| 10-2 (Tris buffer) | 100 μg/ml | 64° | yes |
| 10-2 (surfactant) | 100 μg/ml | 84° | partially |
| 10-4 (surfactant) | 100 μg/ml | 79° | partially |
| 10-5 (surfactant) | 100 μg/ml | 79° | partially |

The surfaces were hydrophilized with all the hydrophobin fusion solutions. In this connection, the effect is most marked using a solution which is buffered but does not comprise any surfactant.

### 2. Spraying Aluminum Sheets

Commercially available aluminum sheets (from Elastogran) were used for the experiments.

In the same way as described above, the aluminum sheets were sprayed with solution 10-1 (only water) or solution 10-2 (hydrophobin fusion in Tris buffer), dried and rinsed with deionized water. The quantity of solution consumed was 150 mL (100 μg/ml) for 1.2 m² of sheeting; this corresponds to about 12.5 mg of hydrophobin/m².

TABLE 2
Coating aluminum plates with hydrophobin fusions

| | Concentration of | Contact | Formation of a |
|---|---|---|---|
| Hydrophobin | hydrophobin | angle | film by water |
| Comparison, | — | 73° | no |
| uncoated | | | |
| aluminum | | | |
| 10-1 (water) | 100 μg/mL | 84° | no, only |
| | (chromatog.) | | punctately |
| 10-2 (Tris | 100 μg/mL | 80° | yes |
| buffer) | (chromatog.) | | |
| 10-6 (water) | 100 μg/mL | 84° | no, only |
| | (disrupted | | punctately |
| | material) | | |
| 10-7 (Tris | 100 μg/mL | 81° | yes |
| buffer) | (disrupted | | |
| | material) | | |

Contact angle measurement shows that the aluminum surface is slightly hydrophobized. A marked modification can be seen with regard to the ability of water to form a film on the aluminum surface.

The two hydrophobin fusion solutions which are worked up in different ways do not differ in regard to their efficacy.

### EXAMPLE 13

### Crosslinking Surfaces and Hydrophobin

### Substrate: Leather (Wet Blue)

Spray-dried hydrophobin is taken up in water and adjusted to a concentration of 100 μg/mL
Leather pieces are incubated overnight (room temperature) in 50 mM Tris, pH 8+0.1 mM CaCl₂ (final concentration)+0.1% polyoxyethylene(20)sorbitan monolaurate (Tween® 20)
After that, the coating is washed in distilled water
After that, an incubation is carried out, at 80° C. for 10 min, in 1% sodium dodecyl sulfate (SDS) solution in dist. water
Washing is carried out in dist. water
An incubation is carried out with a 0.01% solution of glutaraldehyde in water (2 hours at room temperature)
Washing is carried out in dist. water

The leather is hydrophilized to a significant extent and gains additional mechanical stability as a result of the crosslinking. The hydrophilization can be determined in a known manner by means of water drop uptake. While a water drop required approximately 4 min on untreated leather before it had soaked in, a water drop of the same size soaked into the hydrophobin-treated leather within less than 1 min.

### EXAMPLE 15

### Drying by Means of IR Radiation

### Substrate:

Glass (window glass, Süddeutsche Glas, Mannheim):
Hydrophobin which has been spray dried as described in Example 9 is taken up in 10 mM Tris, pH 8, and adjusted to a concentration of 50 μg/mL.
Small glass plates are wetted with the hydrophobin solution and dried within 10 min by means of IR radiation (Philips IR125R). Temperature at the surface from approx. 100 to 120° C.

The contact angle (in degrees) of a 5 μl drop of water is determined at room temperature.

The contact angle was measured on a Dataphysics Contact Angle System OCA 15+, software SCA 20.2.0. (November 2002), appliance. The measurement was carried out in accordance with the manufacturer's instructions.

Untreated glass gave a contact angle of 30±5°; the treated glass gave a contact angle of 75±15°.

Assignment of the sequence names to DNA and polypeptide sequences in the sequence listing

| | |
|---|---|
| dewA DNA and polypeptide sequence | SEQ ID NO: 1 |
| dewA polypeptide sequence | SEQ ID NO: 2 |
| rodA DNA and polypeptide sequence | SEQ ID NO: 3 |
| rodA polypeptide sequence | SEQ ID NO: 4 |
| hypA DNA and polypeptide sequence | SEQ ID NO: 5 |
| hypA polypeptide sequence | SEQ ID NO: 6 |
| hypB DNA and polypeptide sequence | SEQ ID NO: 7 |
| hypB polypeptide sequence | SEQ ID NO: 8 |
| sc3 DNA and polypeptide sequence | SEQ ID NO: 9 |
| sc3 polypeptide sequence | SEQ ID NO: 10 |
| basf1 DNA and polypeptide sequence | SEQ ID NO: 11 |
| basf1 polypeptide sequence | SEQ ID NO: 12 |
| basf2 DNA and polypeptide sequence | SEQ ID NO: 13 |
| basf2 polypeptide sequence | SEQ ID NO: 14 |
| yaad DNA and polypeptide sequence | SEQ ID NO: 15 |
| yaad polypeptide sequence | SEQ ID NO: 16 |
| yaae DNA and polypeptide sequence | SEQ ID NO: 17 |
| yaae polypeptide sequence | SEQ ID NO: 18 |
| yaad-Xa-dewA-his DNA and polypeptide | SEQ ID NO: 19 |
| sequence | |
| yaad-Xa-dewA-his polypeptide sequence | SEQ ID NO: 20 |
| yaad-Xa-rodA-his DNA and polypeptide | SEQ ID NO: 21 |
| sequence | |
| yaad-Xa-rodA-his polypeptide sequence | SEQ ID NO: 22 |
| yaad-Xa-basf1-his DNA and polypeptide | SEQ ID NO: 23 |
| sequence | |
| yaad-Xa-basf1-his polypeptide sequence | SEQ ID NO: 24 |

1. A method for coating surfaces with hydrophobins, comprising at least the following procedural steps:
(1) providing a formulation (F) comprising water or an aqueous solvent mixture and a hydrophobin,
(2) treating the surface with the formulation, and
(3) removing the solvent,
wherein the hydrophobin is a hydrophobin fusion in which a naturally occurring hydrophobin is linked to a peptide sequence which is at least 20 amino acids in length and which is not naturally linked to a hydrophobin and the formulation has a pH of ≧4.

2. The method according to claim 1, wherein the hydrophobin fusion exhibits the structural formula (I)
Xₙ—C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I),
where X can in each case be identical or different and can be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu and Gly), the indices at each X constitute the number of amino acids, C is cysteine, alanine, serine, glycine, methionine or threonine, with at least four of the residues designated by C being cysteine, and the indices n and m are, independently of each other, natural numbers of from 0 to 500, with the proviso that at least one of the peptide sequences designated by Xₙ and Xₘ is a peptide sequence which is at least 20 amino acids in length and which is not naturally linked to a hydrophobin, and also with the further proviso that the polypeptides are characterized by the property that, at room temperature and after having coated a glass surface, they increase the contact angle of a water drop by at least 20°, in each case compared with the contact angle of a water drop of the same size with the uncoated glass surface.

3. The method according to claim 2, wherein the hydrophobin fusion exhibits the following structural formula (III):
Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III),
where the indices n and m stand for numbers between 0 and 200, with the proviso that at least one of the peptide sequences designated by Xₙ and Xₘ is a peptide sequence which is at least 20 amino acids in length and which is not naturally linked to a hydrophobin, and at least 6 of the residues designated by C are cysteine.

4. The method according to claim 3, wherein all of the C radicals are cysteine.

5. The method according claim 1, wherein the fusion partner for the natural hydrophobins is yaad (SEQ ID No: 15 or 16) or fragments or derivatives thereof, yaae (SEQ ID No: 17 or 18) or fragments or derivatives thereof, or thioredoxin, or fragments or derivatives thereof.

6. The method according to claim 1, wherein the hydrophobin fusion exhibits an affinity domain in addition to the fusion partner as a group Xₙ or Xₘ.

7. The method according to claim 6, wherein the affinity domain is a (His)ₖ group, where k is from 4 to 6.

8. The method according to claim 1, wherein the formulation has a pH of ≧7.

9. The method according to claim 1, wherein the formulation additionally comprises a buffer.

10. The method according to claim 1, wherein the formulation is obtained by dissolving solid hydrophobin fusion.

11. The method according to claim 10, wherein the hydrophobin fusion is a spray-dried hydrophobin fusion.

12. The method according to claim 1, wherein use is made, for preparing the formulation, of a solution which is prepared by separating off the cells from the fermentation broth, disrupting the cells and dissolving the inclusion bodies.

13. The method according to claim 1, wherein the coating is performed at from 15 to 120° C.

14. The method according to claim 1, wherein the coating is performed at from 20 to 100° C.

15. The method according to claim 1, wherein the drying is performed at 30-130° C.

16. The method according to claim 1, wherein the coating is crosslinked in an additional procedural step.

17. The method according to claim 1, wherein the hydrophobin fusion is yaad-Xa-dewA-his6 (SEQ ID NO: 20) or a protein comprising a truncated yaad fusion partner.

18. A surface, comprising a coating which comprises at least one hydrophobin fusion in which a naturally occurring hydrophobin is linked to a peptide sequence which is at least 20 amino acids in length and which is not naturally linked to a hydrophobin.

19. The surface according to claim 18, wherein the coating is crosslinked.

20. The surface according to claim 18, wherein the hydrophobin fusion is yaad-Xa-dewA-his (SEQ ID NO: 20) or a protein comprising a truncated yaad fusion partner.
